(19) United States
(12) Patent Application Publication
(10) Pub. No.: US 2010/0105729 A1
(43) Pub. Date: Apr. 29, 2010

(54) ARYL-SUBSTITUTED HETEROCYCLIC PDE4 INHIBITORS AS ANTI-INFLAMMATORY AGENTS

(75) Inventors: Steven P. Govek, San Diego, CA (US); Guy Oshiro, San Diego, CA (US); Stewart A. Noble, San Diego, CA (US); James W. Malecha, San Diego, CA (US); Andrew K. Shiau, San Diego, CA (US)

Correspondence Address: GLOBAL PATENT GROUP - KAL, 10411 Clayton Road, Suite 304, St. Louis, MO, 63131, US

(73) Assignee: KALYPSYS, INC., San Diego, CA (US)

(21) Appl. No.: 12/444,533

(22) PCT Filed: Sep. 20, 2007
(86) PCT No.: PCT/US07/78980

§ 371 Date: May 12, 2009

Related U.S. Application Data
(60) Provisional application No. 60/850,117, filed on Oct. 6, 2006.

(51) Int. Cl. A61K 31/4155; A61K 31/4709; C07D 409/04; C07D 401/04; C07D 403/04; A61P 29/00

(52) U.S. Cl. 514/314; 514/403; 514/406; 548/365.7; 546/167; 548/365.4

(57) ABSTRACT

Disclosed herein are potent and selective aryl-substituted heterocyclic compounds, useful as inhibitors of phosphodiesterase 4 (PDE4), compositions comprising the same, and their application as pharmaceuticals for the treatment of disease. Methods of inhibition of PDE4 activity are also provided, as well as methods for the treatment of inflammatory diseases and other diseases in which PDE4 or one of its isoforms may play a role.

This application claims the benefit of U.S. Provisional Application No. 60/850,117, filed Oct. 6, 2006, the disclosure of which is hereby incorporated by reference as if written herein in its entirety.

Disclosed herein are potent and selective new aryl-substituted heterocyclic compounds, certain of which have been found to be useful as inhibitors of phosphodiesterase 4 (PDE4), compositions comprising the same, and their application as pharmaceuticals for the treatment of disease. Methods of inhibition of PDE4 activity are also provided, as well as methods for the treatment of inflammatory diseases and other diseases involving elevated levels of cytokines and proinflammatory mediators.

Chronic inflammation is a multi-factorial disease complication characterized by activation of multiple types of inflammatory cells, for example cells of lymphoid lineage (including T lymphocytes) and myeloid lineage (including granulocytes, macrophages, and monocytes). Proinflammatory mediators, including cytokines, such as tumor necrosis factor (TNF) and interleukin-1 (IL-1), are produced by these activated cells. Accordingly, an agent that suppresses the activation of these cells, or their production of proinflammatory cytokines, would be useful in the therapeutic treatment of inflammatory diseases and other diseases involving elevated levels of cytokines.

Cyclic adenosine monophosphate (cAMP) is a second messenger that mediates the biologic responses of cells to a wide range of extracellular stimuli. When the appropriate agonist binds to specific cell surface receptors, adenylate cyclase is activated to convert adenosine triphosphate (ATP) to cAMP. It is theorized that the agonist induced actions of cAMP within the cell are mediated predominately by the action of cAMP-dependent protein kinases. The intracellular actions of cAMP are terminated by either a transport of the nucleotide to the outside of the cell, or by enzymatic cleavage by cyclic nucleotide phosphodiesterases (PDEs), which hydrolyze the 3′-phosphodiester bond to form 5′-adenosine monophosphate (5′-AMP). 5′-AMP is an inactive metabolite.

The superfamily of PDEs is subdivided into two major classes, class I and class II, which have no recognizable sequence similarity. Class I includes all known mammalian PDEs and is comprised of 11 identified families that are products of separate genes. Some PDEs are highly specific for hydrolysis of cAMP (PDE4, PDE7, PDE8), some are highly cGMP-specific (PDE5, PDE6, PDE9), and some have mixed specificity (PDE1, PDE2, PDE3, PDE10, PDE11). All of the characterized mammalian PDEs are dimeric, but the importance of the dimeric structure for function in each of the PDEs is unknown.

The PDE4 subfamily is comprised of 4 members: PDE4A, PDE4B, PDE4C, and PDE4D. These enzymes possess N-terminal regulatory domains that presumably mediate dimerization, which results in optimally regulated PDE activity. In addition, activity is regulated via cAMP-dependent protein kinase phosphorylation sites in this upstream regulatory domain. PDE4 enzymes are broadly expressed and distributed.

Elevated levels of cAMP in human myeloid and lymphoid lineage cells are associated with the suppression of cell activation. The intracellular enzyme family of PDEs, therefore, regulates the level of cAMP in cells. PDE4 is a predominant PDE isotype in these cells, and is a major contributor to cAMP degradation. Accordingly, the inhibition of PDE function would prevent the conversion of cAMP to the inactive metabolite 5′-AMP and, consequently, maintain higher cAMP levels, and, accordingly, suppress cell activation.

PDE4 inhibitors have been shown to inhibit production of TNFα and partially inhibit IL-1β release by monocytes (see Semmler et al., Int. J. Immunopharmacol., 15, pp. 409-413, (1993); Molnar-Kimber et al., Mediators of Inflammation, 1, pp. 411-417, (1992)). PDE4 inhibitors also have been shown to inhibit the production of superoxide radicals from human polymorphonuclear leukocytes (see Verghese et al., J. Mol. Cell. Cardiol., 21 (Suppl. 2), S61 (1989); Nielson et al., J. Allergy Immunol., 86, pp. 801-808, (1990)); to inhibit the release of vasoactive amines and prostanoids from human basophils (see Peachell et al., J. Immunol., 148, pp. 2503-2510, (1992)); to inhibit respiratory bursts in eosinophils (see Dent et al., J. Pharmacol., 103, pp. 1339-1346, (1991)); and to inhibit the activation of human T-lymphocytes (see Robicsek et al., Biochem. Pharmacol., 42, pp. 869-877, (1991)).

Inflammatory cell activation and excessive or unregulated cytokine (e.g., TNFα and IL-1β) production are implicated in allergic, autoimmune, and inflammatory diseases and disorders, such as rheumatoid arthritis, osteoarthritis, gouty arthritis, spondylitis, thyroid associated opthalmopathy, Behcet's disease, sepsis, septic shock, endotoxic shock, gram negative sepsis, gram positive sepsis, toxic shock syndrome, asthma, chronic bronchitis, adult respiratory distress syndrome, chronic pulmonary inflammatory disease, such as chronic obstructive pulmonary disease, silicosis, pulmonary sarcoidosis, reperfusion injury of the myocardium, brain, and extremities, fibrosis, cystic fibrosis, keloid formation, scar formation, atherosclerosis, transplant rejection disorders, such as graft vs. host reaction and allograft rejection, chronic glomerulonephritis, lupus, inflammatory bowel disease, such as Crohn's disease and ulcerative colitis, proliferative lymphocyte diseases, such as leukemia, opthalmologic diseases such as dry eye and ocular pain resulting from inflammation or surgery, and inflammatory dermatoses, such as atopic dermatitis, psoriasis, and urticaria.

Other conditions characterized by elevated cytokine levels include brain injury due to moderate trauma, cardiomyopathies, such as congestive heart failure, cachexia, cachexia secondary to infection or malignancy, cachexia secondary to acquired immune deficiency syndrome (AIDS), ARC (AIDS related complex), fever myalgias due to infection, cerebral malaria, osteoporosis and bone resorption diseases, keloid formation, scar tissue formation, and pyrexia.

Additionally, several properties of TNFα, such as stimulation of collagenases, stimulation of angiogenesis in vivo, stimulation of bone resorption, and an ability to increase the adherence of tumor cells to endothelium, are consistent with a role for TNF in the development and metastatic spread of cancer in the host. TNFα recently has been directly implicated in the promotion of growth and metastasis of tumor cells (see Orosz et al., J. Exp. Med., 177, pp. 1391-1398, (1993)).

Investigators have shown considerable interest in the use of PDE4 inhibitors as anti-inflammatory agents. Early evidence indicates that PDE4 inhibition has beneficial effects on a variety of inflammatory cells such as monocytes, macrophages, T-cells of the Th-1 lineage, and granulocytes. The synthesis and/or release of many proinflammatory mediators, such as cytokines, lipid mediators, superoxide, and biogenic amines, such as histamine, have been attenuated in these cells by the action of PDE4 inhibitors. The PDE4 inhibitors also affect other cellular functions including T-cell proliferation, granulocyte transmigration in response to chemotoxic substances, and integrity of endothelial cell junctions within the vasculature.

The design, synthesis, and screening of various PDE4 inhibitors have been reported. Methylxanthines, such as caffeine and theophylline, were the first PDE inhibitors discovered, but these compounds are nonselective with respect to which PDE is inhibited. The drug rolipram, an antidepressant agent, was one of the first reported specific PDE4 inhibitors, with a reported IC₅₀ of about 200 nM with respect to inhibiting recombinant human PDE4.

Investigators have continued to search for PDE4 inhibitors that are more selective with respect to inhibiting PDE4, that have a lower IC₅₀ than rolipram, and that avoid the undesirable central nervous system (CNS) side effects, such as retching, vomiting, and sedation, associated with the administration of rolipram. In addition, several companies are now undertaking clinical trials of other PDE4 inhibitors. However, problems relating to efficacy and adverse side effects, such as emesis and central nervous system disturbances, remain unsolved.

Accordingly, compounds that selectively inhibit PDE4, and that reduce or eliminate the adverse side effects associated with prior PDE4 inhibitors, would be useful in the treatment of allergic and inflammatory diseases, and other diseases associated with excessive or unregulated production of cytokines, such as TNF. In addition, selective PDE4 inhibitors would be useful in the treatment of diseases that are associated with elevated cAMP levels or PDE4 function in a particular target tissue.

Novel compounds and pharmaceutical compositions useful as anti-inflammatory agents via the inhibition of PDE4 have been found, together with methods of synthesizing and using the compounds including methods for inhibiting PDE4 in a patient by administering the compounds.

Disclosed herein is a class of compounds, certain of which have been found to be useful in treating PDE4-mediated disorders and conditions, defined by structural Formula I

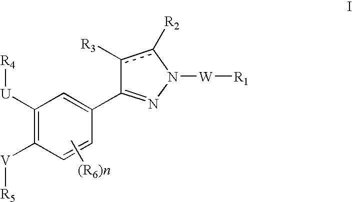

or a salt, ester, or prodrug thereof, wherein:

W is selected from the group consisting of lower alkylene, C(═O), S(═O), SO₂, and null;

U and V are each independently selected from the group consisting of O, S, NR⁷, S(═O), and SO₂;

R¹ is selected from the group consisting of hydrogen, alkenyl, alkoxy, alkyl, alkynyl, amino, aryl, aryloxy, carboxyalkyl, cycloalkoxy, cycloalkyl, heteroaryl, heterocycloalkoxy, heterocycloalkyl, and mercaptyl, any of which may be optionally substituted;

R² is selected from the group consisting of acyl, alkenyl, alkyl, alkynyl, amido, aryl, carboxyl, cyano, cycloalkyl, ester, heteroaryl, heterocycloalkyl, sulfonate, sulfinyl, sulfonyl, and S-sulfonamido, any of which may be optionally substituted;

R³ is selected from the group consisting of hydrogen, acyl, acylamino, acyloxy, alkoxy, alkyl, amino, N-carbamyl, O-carbamyl, hydroxy, and mercaptyl, any of which may be optionally substituted;

R⁴ is selected from the group consisting of acyl, alkenyl, alkyl, alkynyl, aryl, cycloalkyl, cycloalkylalkyl, heteroaryl, and heterocycloalkyl, any of which may be optionally substituted;

R⁵ is selected from the group consisting of alkyl, haloalkyl, and perhaloalkyl, any of which may be optionally substituted;

each R⁶ is independently selected from the group consisting of alkoxy, alkyl, cycloalkoxy, halogen, hydroxy, mercaptyl, nitro, sulfonate, sulfinyl, and sulfonyl, any of which may be optionally substituted;

R⁷ is selected from the group consisting of hydrogen and optionally substituted lower alkyl; and

n is an integer from 0 to 3.

Certain compounds disclosed herein may possess useful PDE4 inhibiting activity, and may be used in the treatment or prophylaxis of a disease or condition in which PDE plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting PDE4. Other embodiments provide methods for treating a PDE4-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of PDE4.

In further embodiments, compounds of the present invention have structural Formula II

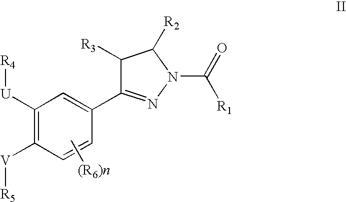

or a salt, ester, or prodrug thereof, wherein:

U and V are each independently selected from the group consisting of O and S;

R¹ is selected from the group consisting of hydrogen, alkenyl, alkyl, alkynyl, aryl, carboxyalkyl, cycloalkyl, heteroaryl, and heterocycloalkyl, any of which may be optionally substituted;

R² is selected from the group consisting of aryl and heteroaryl, either of which may be optionally substituted;

R³ is selected from the group consisting of hydrogen and optionally substituted lower alkyl;

R⁴ and R⁵ are independently selected from the group consisting of alkyl, haloalkyl, and perhaloalkyl, or R⁴ and R⁵ may combine to form a heterocyclic ring system selected from the group consisting of 1,3-dioxolane, 1,4-dioxane, 1,3-oxathiolane, 1,4-oxathiane, 1,3-dithiolane, and 1,4-dithiane, any of which may be optionally substituted by lower alkyl or halogen;

each R⁶ is independently selected from the group consisting of alkoxy, alkyl, halogen, hydroxy, and mercaptyl, any of which may be optionally substituted; and

n is an integer from 0 to 3.

In yet further embodiments, R² is optionally substituted phenyl.

In certain embodiments, the compounds of the present invention have structural Formula III

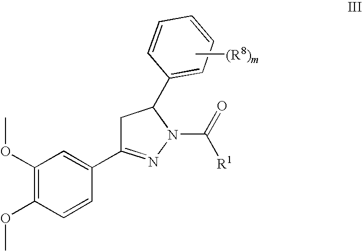

or a salt, ester, or prodrug thereof, wherein:

R¹ is selected from the group consisting of alkyl, carboxyalkyl, heteroaryl, and aryl, any of which may be optionally substituted;

each R⁸ is independently selected from the group consisting of alkoxy, halogen, amino, nitro and alkyl, any of which may be optionally substituted; and

m is an integer from 0 to 2.

Compounds of the present invention may be selective amongst the PDE4 isoforms PDE4A, PDE4B, PDE4C, and PDE4D in various ways. For example, compounds described herein may be selective for PDE4B and PDE4D over the other two isoforms, be a pan-inhibitor of all the isoforms, or be selective for only one isoform. In certain embodiments, compounds of the present invention may be selective for PDE4B over other isoforms.

Certain embodiments of the present invention also relate to a method of inhibiting at least one PDE4 function comprising the step of contacting the PDE4 with a compound of Formula I, as described herein. The cell phenotype, cell proliferation, activity of PDE4, change in biochemical output produced by active PDE4, expression of PDE4, or binding of PDE4 with a natural binding partner may be monitored. Such methods may be modes of treatment of disease, biological assays, cellular assays, biochemical assays, or the like.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether

(R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group—with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene

(—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups may be exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—. The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphoamide” as used herein, alone or in combination, refers to a phosphate group [(OH)₂P(O)O—] in which one or more of the hydroxyl groups has been replaced by nitrogen, amino, or amido.

The term “phosphonate” as used herein, alone or in combination, refers to a group of the form ROP(OR′)(OR)O— wherein R and R′ are selected from the group consisting of hydrogen, alkyl, acyl, heteroalkyl, aryl, cycloalkyl, heteroaryl, and heterocycloalkyl, any of which may themselves be optionally substituted. “Phosphonate” includes “phosphate [(OH)₂P(O)O—] and related phosphoric acid anions which may form salts.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —SO₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PDE4 inhibitor” is used herein to refer to a compound that exhibits an IC₅₀ with respect to PDE4 activity of no more than about 100 μM and more typically not more than about 50 μM, as measured in the PDE4 assay described generally hereinbelow. “IC₅₀” is that concentration of inhibitor which reduces the activity of an enzyme (e.g., PDE4) to half-maximal level. Certain compounds disclosed herein have been discovered to exhibit inhibition against PDE4. In certain embodiments, compounds will exhibit an IC₅₀ with respect to PDE4 of no more than about 10 μM; in further embodiments, compounds will exhibit an IC₅₀ with respect to PDE4 of no more than about 5 μM; in yet further embodiments, compounds will exhibit an IC₅₀ with respect to PDE4 of not more than about 1 μM, as measured in the PDE4 assay described herein. In yet further embodiments, compounds will exhibit an IC₅₀ with respect to PDE4 of not more than about 200 nM.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents.

Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for dry eye involving administration of one of the compounds described herein, increased therapeutic benefit may result by also providing the patient with another therapeutic agent for dry eye. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating PDE4-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of PDE4-mediated disorders.

Compounds of the subject invention may be useful in treating PDE4-mediated disease, disorders and conditions. In certain embodiments, said compounds may find use in treating acute and chronic pain and inflammation. The compounds of the present invention may be useful to treat patients with neuropathy, neuropathic pain, or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The compounds may also be useful in the treatment of pain associated with acute herpes zoster (shingles), postherpetic neuralgia (PHN), and associated pain syndromes such as ocular pain. The compounds may further be useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like. The compounds may also be useful for the treatment of pain-related disorders such as tactile allodynia and hyperalgesia. The pain may be somatogenic (either nociceptive or neuropathic), acute and/or chronic. The PDE4 inhibitors of the subject invention may also be useful in conditions where NSAIDs, morphine or fentanyl opiates and/or other opioid analgesics would traditionally be administered.

Furthermore, compounds of the subject invention may be used in the treatment or prevention of opiate tolerance in patients needing protracted opiate analgesics, and benzodiazepine tolerance in patients taking benzodiazepines, and other addictive behavior, for example, nicotine addiction, alcoholism, and eating disorders. Moreover, the compounds and methods of the present invention may be useful in the treatment or prevention of drug withdrawal symptoms, for example treatment or prevention of symptoms of withdrawal from opiate, alcohol, or tobacco addiction.

In addition, compounds of the subject invention may be used to treat insulin resistance and other metabolic disorders such as atherosclerosis that are typically associated with an exaggerated inflammatory signaling.

The present invention encompasses therapeutic methods using novel selective PDE4 inhibitors to treat or prevent respiratory disease or conditions, including therapeutic methods of use in medicine for preventing and treating a respiratory disease or condition including: asthmatic conditions including allergen-induced asthma, exercise-induced asthma, pollution-induced asthma, cold-induced asthma, and viral-induced-asthma; asthma-related diseases such as airway hyperreactivity and small airway disease; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchiolitis, bronchioectasis, cystic fibrosis, pigeon fancier's disease, farmer's lung, acute respiratory distress syndrome, pneumonia, pneumonitis, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus, hypoxia, dyspnea, hypercapnea, hyperinflation, hypoxemia, and cough. Further, compounds disclosed herein would find use in the treatment of allergic disorders such as delayed type hypersensitivity reaction, allergic contact dermatitis, allergic rhinitis, and chronic sinusitis.

Other disorders or conditions which may be treated by the compounds of the present invention include inflammation and related disorders. The compounds of the present invention may be useful as anti-inflammatory agents with the additional benefit of having significantly less harmful side effects. The compounds may be useful to treat arthritis, including but not limited to rheumatoid arthritis, spondyloarthropathies, gouty arthritis, osteoarthritis, juvenile arthritis, acute rheumatic arthritis, enteropathic arthritis, neuropathic arthritis, psoriatic arthritis, reactive arthritis (Reiter's syndrome), and pyogenic arthritis, and autoimmune diseases, including systemic lupus erythematosus, hemolytic syndromes, autoimmune hepatitis, autoimmune neuropathy, vitiglio (autoimmune thyroiditis), Hashimoto's thyroiditis, anemias, myositis including polymyositis, alopecia greata, Goodpasture's syndrome, hypophytis, and pulmonary fibrosis.

The compounds may also be useful in treating osteoporosis and other related bone disorders.

These compounds may also be used to treat gastrointestinal conditions such as reflux esophagitis, diarrhea, inflammatory bowel disease, Crohn's disease, gastritis, irritable bowel syndrome, Graves' disease (hyperthyroidism), necrotizing enterocolitis, and ulcerative colitis. The compounds may also be used in the treatment of pulmonary inflammation, such as that associated with viral infections and cystic fibrosis.

In addition, compounds of invention may also be useful in organ transplant patients either alone or in combination with conventional immunomodulators. Examples of conditions to be treated in said patients include graft vs. host reaction (i.e., graft vs. host disease), allograft rejections (e.g., acute allograft rejection, and chronic allograft rejection), transplant reperfusion injury, and early transplantation rejection (e.g., acute allograft rejection).

Yet further, the compounds of the invention may be useful in the treatment of pruritis and vitaligo.

The compounds of the present invention may also be useful in treating tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Langerhans' cell histiocytosis, glomerulonephritis, reperfusion injury, pancreatitis, interstitial cystitis, Behcet's syndrome, polymyositis, gingivitis, periodontis, hypersensitivity, swelling occurring after injury, ischemias including myocardial ischemia, cardiovascular ischemia, and ischemia secondary to cardiac arrest, cirrhosis, septic shock, endotoxic shock, gram negative sepsis, toxic shock syndrome, stroke, ischemia reperfusion injury, multi-organ dysfunction, restenosis including restenosis following coronary bypass surgery, and the like.

The compounds of the subject invention may also be useful for the treatment of certain diseases and disorders of the nervous system. Central nervous system disorders in which PDE4 inhibition may be useful include cortical dementias including Alzheimer's disease and mild cognitive impairment (MCI), central nervous system damage resulting from stroke, ischemias including cerebral ischemia (both focal ischemia, thrombotic stroke and global ischemia (for example, secondary to cardiac arrest), and trauma. Neurodegenerative disorders in which PDE4 inhibition may be useful include nerve degeneration or nerve necrosis in disorders such as hypoxia, hypoglycemia, epilepsy, and in cases of central nervous system (CNS) trauma (such as spinal cord and head injury), hyperbaric oxygen convulsions and toxicity, dementia e.g. pre-senile dementia, and AIDS-related dementia, cachexia, Sydenham's chorea, Huntington's disease, Parkinson's Disease, amyotrophic lateral sclerosis (ALS), Korsakoff's syndrome, and imbecility relating to a cerebral vessel disorder. Further disorders in which PDE4 inhibition might prove useful include neuropathies of the central and peripheral nervous system (including, for example, IgA neuropathy, membranous neuropathy and idiopathic neuropathy), chronic inflammatory demyelinating polyneuropathy, transverse myelitis, Gullain-Barre disease, encephalitis, and cancers of the nervous system. Disorders of CNS function in which PDE4 inhibitors may find use include sleeping disorders, schizophrenia, depression, depression or other symptoms associated with Premenstrual Syndrome (PMS), and anxiety.

Furthermore, the compounds of the present invention may also be useful in inhibiting PDE4 activity for the amelioration of systemic disorders including systemic hypotension associated with septic and/or toxic hemorrhagic shock induced by a wide variety of agents; as a therapy with cytokines such as TNF, IL-1 and IL-2; and as an adjuvant to short term immunosuppression in transplant therapy.

Still other disorders or conditions which may be treated by the compounds of the subject invention include the prevention or treatment of cancer, such as colorectal cancer, and cancer of the breast, lung, prostate, bladder, cervix and skin. Compounds of the invention may be used in the treatment and prevention of neoplasias including but not limited to brain cancer, bone cancer, leukemia, lymphoma, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. The neoplasia can be selected from gastrointestinal cancer, liver cancer, bladder cancer, pancreas cancer, ovary cancer, prostate cancer, cervical cancer, lung cancer, breast cancer and skin cancer, such as squamous cell and basal cell cancers. The present compounds and methods may also be used to treat the fibrosis which occurs with radiation therapy. The present compounds and methods may be used to treat subjects having adenomatous polyps, including those with familial adenomatous polyposis (FAP). Additionally, the present compounds and methods may be used to prevent polyps from forming in patients at risk of FAP.

The compounds of the subject invention may be used in the treatment of ophthalmic diseases, such as dry eye, glaucoma, corneal neovascularization, optic neuritis, Sjogren's syndrome, retinal ganglion degeneration, ocular ischemia, retinitis, retinopathies, uveitis, ocular photophobia, and of inflammation and pain associated with acute injury to the eye tissue. Specifically, the compounds may be used to treat glaucomatous retinopathy and/or diabetic retinopathy. The compounds may also be used to treat post-operative inflammation or pain as from ophthalmic surgery such as cataract surgery and refractive surgery.

Moreover, compounds of the subject invention may be used in the treatment of menstrual cramps, dysmenorrhea, premature labor, endometriosis, tendonitis, bursitis, skin-related conditions such as psoriasis, eczema, burns, sunburn, dermatitis, pancreatitis, hepatitis, lichen planus, scleritis, scleroderma, dermatomyositis, and the like. Other conditions in which the compounds of the subject invention may be used include diabetes (type I or type II), atherosclerosis, congestive heart failure, myocarditis, atherosclerosis, cerebral ischemia, angiogenesis, pulmonary hypertension, and aortic aneurysm.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds of the subject invention may also be used to prevent tissue damage when therapeutically combined with antibacterial or antiviral agents.

Besides being useful for human treatment, compounds and formulations disclosed herein may also be useful for veterinary treatment of companion animals, exotic animals and farm animals, including mammals, rodents, and the like. More preferred animals include horses, dogs, and cats.

The invention is further illustrated by the following examples. Compounds according to the present invention, including Examples 1-106 listed below, can be made by methods known in the art. Additionally, Examples 1-106 may be commercially available. All IUPAC names were generated using CambridgeSoft's ChemDraw 10.0.

### EXAMPLE 1

### 5-(3-(3,4-dimethoxyphenyl)-5-(thiophen-2-yl)-4,5-dihydro-1H-pyrazol-1-yl)-5-oxopentanoic acid

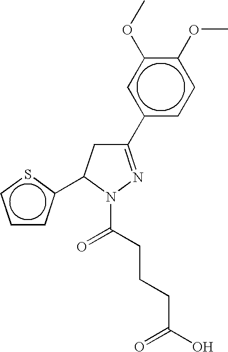

### EXAMPLE 2

### 3-(3,4-dimethoxyphenyl)-1-(methylsulfonyl)-5-(thiophen-2-yl)-4,5-dihydro-1H-pyrazole

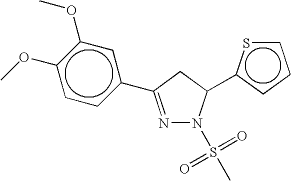

### EXAMPLE 3

### 5-(2-chloro-6-fluorophenyl)-3-(3,4-dimethoxyphenyl)-1-(methylsulfonyl)-4,5-dihydro-1H-pyrazole

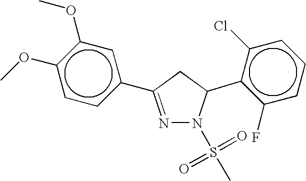

### EXAMPLE 4

### 3-(3,4-dimethoxyphenyl)-5-(3-methoxyphenyl)-1-(methylsulfonyl)-4,5-dihydro-1H-pyrazole

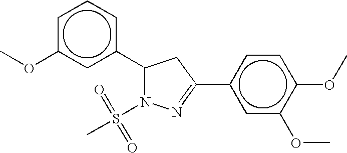

### EXAMPLE 5

### 4-(5-(2-chloro-8-methylquinolin-3-yl)-3-(3,4-dimethoxyphenyl)-4,5-dihydro-1H-pyrazol-1-yl)-4-oxobutanoic acid

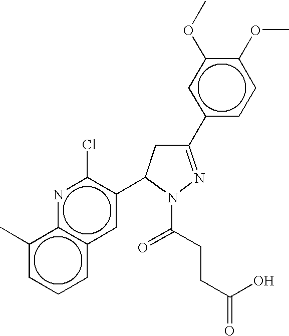

### EXAMPLE 6

### (5-(2-chloro-6-methoxyquinolin-3-yl)-3-(3,4-dimethoxyphenyl)-4,5-dihydro-1H-pyrazol-1-yl)(2-chlorophenyl)methanone

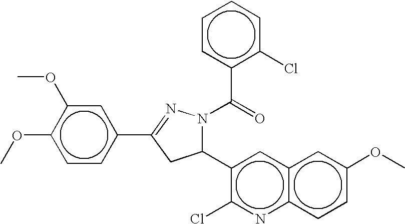

### EXAMPLE 7

### (5-(2-chloro-6-methoxyquinolin-3-yl)-3-(3,4-dimethoxyphenyl)-4,5-dihydro-1H-pyrazol-1-yl)(phenyl)methanone

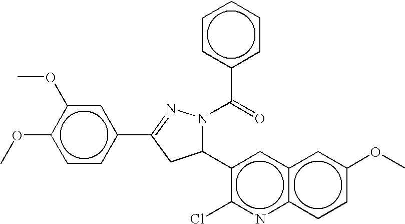

### EXAMPLE 8

### 1-(5-(2-chloro-6-methoxyquinolin-3-yl)-3-(3,4-dimethoxyphenyl)-4,5-dihydro-1H-pyrazol-1-yl)propan-1-one

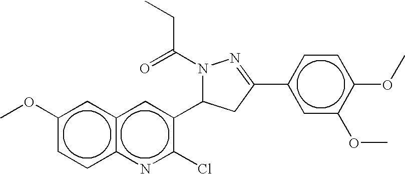

### EXAMPLE 9

### (5-(2-chloro-6-methoxyquinolin-3-yl)-3-(3,4-dimethoxyphenyl)-4,5-dihydro-1H-pyrazol-1-yl)(furan-2-yl)methanone

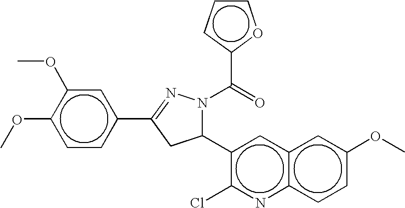

### EXAMPLE 10

### (5-(2-chloro-7-methoxyquinolin-3-yl)-3-(3,4-dimethoxyphenyl)-4,5-dihydro-1H-pyrazol-1-yl)(furan-2-yl)methanone

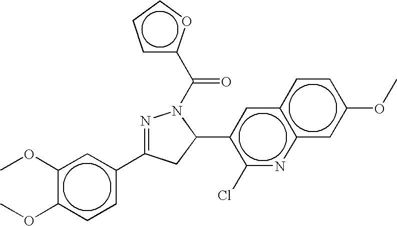

### EXAMPLE 11

### 5-(2,3-dimethoxyphenyl)-3-(3,4-dimethoxyphenyl)-1-(methylsulfonyl)-4,5-dihydro-1H-pyrazole

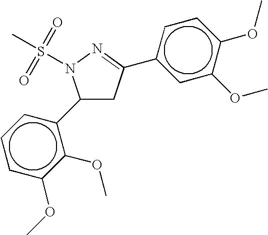

### EXAMPLE 12

### 3-(3,4-dimethoxyphenyl)-5-(4-ethylphenyl)-1-(methylsulfonyl)-4,5-dihydro-1H-pyrazole

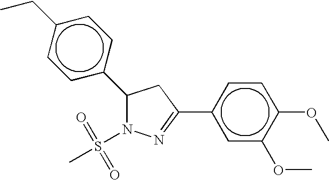

### EXAMPLE 13

### 3-(3,4-dimethoxyphenyl)-1-(methylsulfonyl)-5-p-tolyl-4,5-dihydro-1H-pyrazole

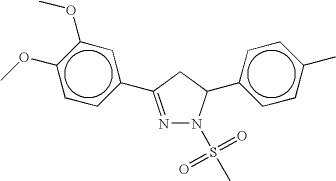

### EXAMPLE 14

### 3-(3,4-dimethoxyphenyl)-5-(4-methoxyphenyl)-1-(methylsulfonyl)-4,5-dihydro-1H-pyrazole

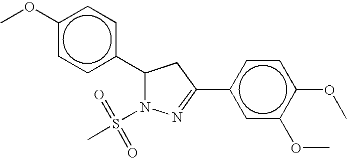

### EXAMPLE 15

### 5-(2-chlorophenyl)-3-(3,4-dimethoxyphenyl)-1-(methylsulfonyl)-4,5-dihydro-1H-pyrazole

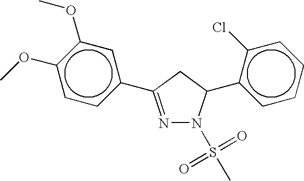

### EXAMPLE 16

### (5-(2-chloro-7-methoxyquinolin-3-yl)-3-(3,4-dimethoxyphenyl)-4,5-dihydro-1H-pyrazol-1-yl)(2-chlorophenyl)methanone

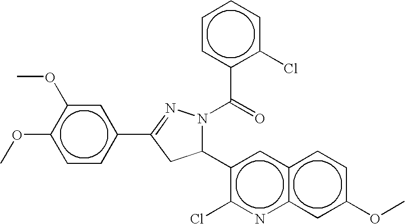

### EXAMPLE 17

### 5-(5-(2-chloro-7-methoxyquinolin-3-yl)-3-(3,4-dimethoxyphenyl)-4,5-dihydro-1H-pyrazol-1-yl)-5-oxopentanoic acid

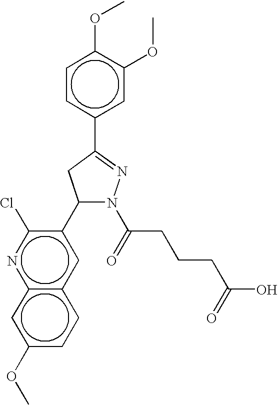

### EXAMPLE 18

### 5-(3-(3,4-dimethoxyphenyl)-5-(4-ethylphenyl)-4,5-dihydro-1H-pyrazol-1-yl)-5-oxopentanoic acid

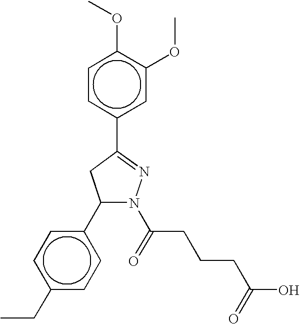

### EXAMPLE 19

### 4-(3-(3,4-dimethoxyphenyl)-5-(4-ethylphenyl)-4,5-dihydro-1H-pyrazol-1-yl)-4-oxobutanoic acid

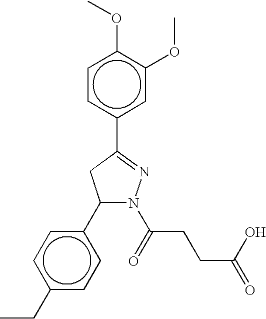

### EXAMPLE 20

### 1-(3-(3,4-dimethoxyphenyl)-5-(4-ethylphenyl)-4,5-dihydro-1H-pyrazol-1-yl)propan-1-one

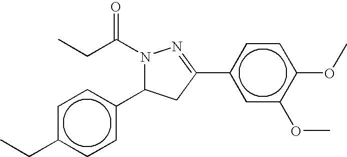

### EXAMPLE 21

### 1-(3-(3,4-dimethoxyphenyl)-5-(4-ethylphenyl)-4,5-dihydro-1H-pyrazol-1-yl)ethanone

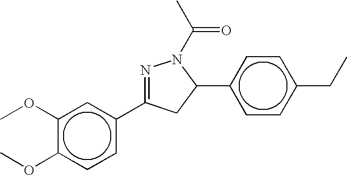

### EXAMPLE 22

### 4-(5-(2-chloro-7-methoxyquinolin-3-yl)-3-(3,4-dimethoxyphenyl)-4,5-dihydro-1H-pyrazol-1-yl)-4-oxobutanoic acid

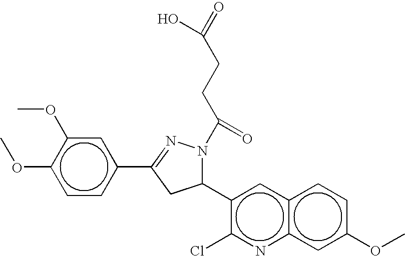

### EXAMPLE 23

### 1-(5-(2-chloro-7-methoxyquinolin-3-yl)-3-(3,4-dimethoxyphenyl)-4,5-dihydro-1H-pyrazol-1-yl)ethanone

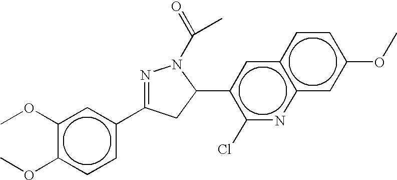

### EXAMPLE 24

### 4-(3-(3,4-dimethoxyphenyl)-1-tosyl-4,5-dihydro-1H-pyrazol-5-yl)-1,3-diphenyl-1H-pyrazole

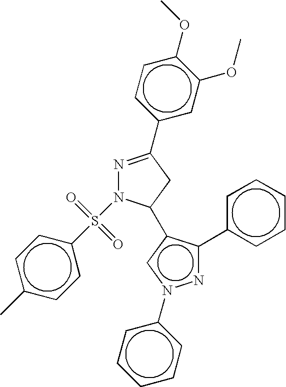

### EXAMPLE 25

### 4-(3-(3,4-dimethoxyphenyl)-1-(phenylsulfonyl)-4,5-dihydro-1H-pyrazol-5-yl)-1,3-diphenyl-1H-pyrazole

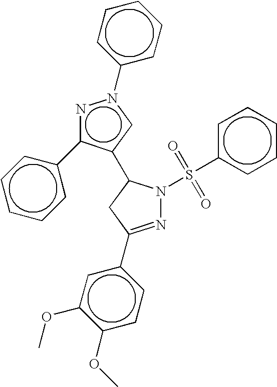

### EXAMPLE 26

### (2-chlorophenyl)(3-(3,4-dimethoxyphenyl)-5-(1,3-diphenyl-1H-pyrazol-4-yl)-4,5-dihydro-1H-pyrazol-1-yl)methanone

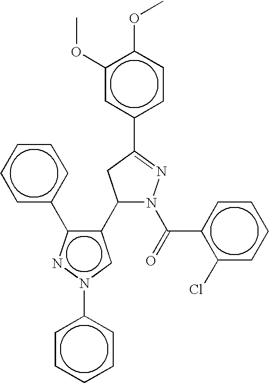

### EXAMPLE 27

### 4-(5-(2-chloroquinolin-3-yl)-3-(3,4-dimethoxyphenyl)-4,5-dihydro-1H-pyrazol-1-yl)-4-oxobutanoic acid

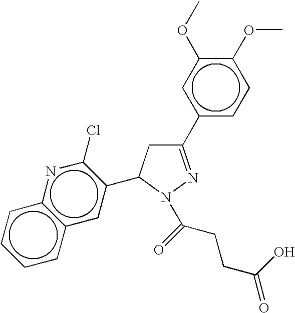

### EXAMPLE 28

### 1-(5-(2-chloroquinolin-3-yl)-3-(3,4-dimethoxyphenyl)-4,5-dihydro-1H-pyrazol-1-yl)ethanone

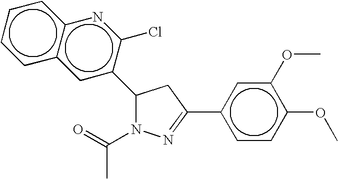

### EXAMPLE 29

### (2-chlorophenyl)(5-(2,5-dimethoxyphenyl)-3-(3,4-dimethoxyphenyl)-4,5-dihydro-1H-pyrazol-1-yl)methanone

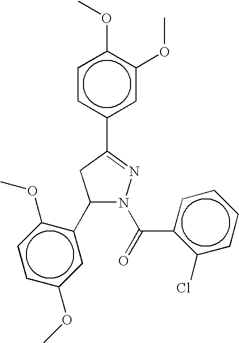

### EXAMPLE 30

### (2-chlorophenyl)(5-(2,3-dimethoxyphenyl)-3-(3,4-dimethoxyphenyl)-4,5-dihydro-1H-pyrazol-1-yl)methanone

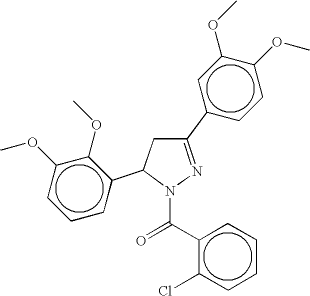

### EXAMPLE 31

### (2-chlorophenyl)(3-(3,4-dimethoxyphenyl)-5-(4-fluorophenyl)-4,5-dihydro-1H-pyrazol-1-yl)methanone

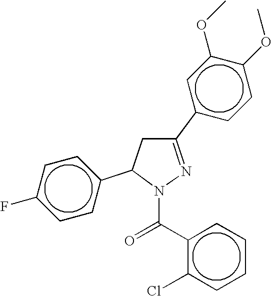

### EXAMPLE 32

### (2-chlorophenyl)(3-(3,4-dimethoxyphenyl)-5-p-tolyl-4,5-dihydro-1H-pyrazol-1-yl)methanone

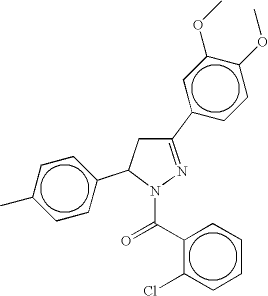

### EXAMPLE 33

### (2-chlorophenyl)(3-(3,4-dimethoxyphenyl)-5-(4-methoxyphenyl)-4,5-dihydro-1H-pyrazol-1-yl)methanone

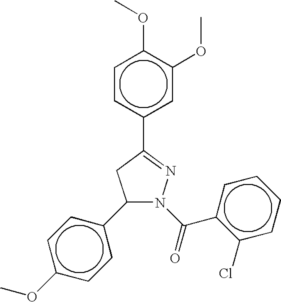

### EXAMPLE 34

### (2-chlorophenyl)(3-(3,4-dimethoxyphenyl)-5-(2-fluorophenyl)-4,5-dihydro-1H-pyrazol-1-yl)methanone

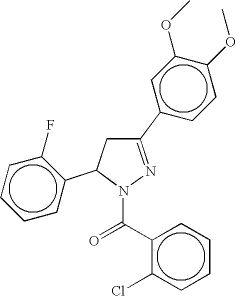

### EXAMPLE 35

### (2-chlorophenyl)(5-(2-chlorophenyl)-3-(3,4-dimethoxyphenyl)-4,5-dihydro-1H-pyrazol-1-yl)methanone

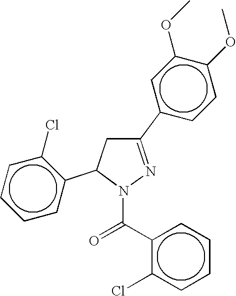

### EXAMPLE 36

### (3-(3,4-dimethoxyphenyl)-5-(4-methoxyphenyl)-4,5-dihydro-1H-pyrazol-1-yl)(phenyl)methanone

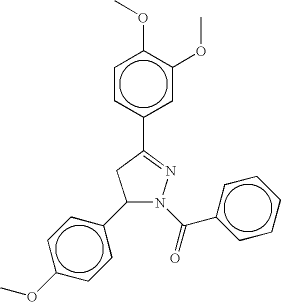

### EXAMPLE 37

### (3-(3,4-dimethoxyphenyl)-5-(4-(dimethylamino)phenyl)-4,5-dihydro-1H-pyrazol-1-yl)(phenyl)methanone

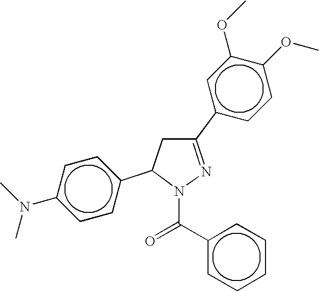

### EXAMPLE 38

### (5-(2,3-dimethoxyphenyl)-3-(3,4-dimethoxyphenyl)-4,5-dihydro-1H-pyrazol-1-yl)(thiophen-2-yl)methanone

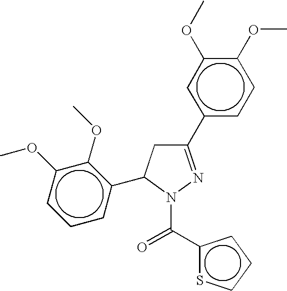

### EXAMPLE 39

### (5-(2-chlorophenyl)-3-(3,4-dimethoxyphenyl)-4,5-dihydro-1H-pyrazol-1-yl)(thiophen-2-yl)methanone

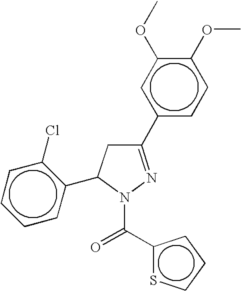

### EXAMPLE 40

### (3-(3,4-dimethoxyphenyl)-5-(2-fluorophenyl)-4,5-dihydro-1H-pyrazol-1-yl)(thiophen-2-yl)methanone

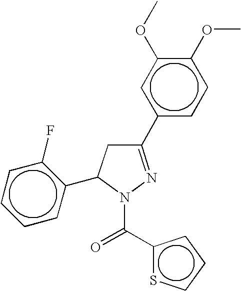

### EXAMPLE 41

### (3-(3,4-dimethoxyphenyl)-5-(4-methoxyphenyl)-4,5-dihydro-1H-pyrazol-1-yl)(thiophen-2-yl)methanone

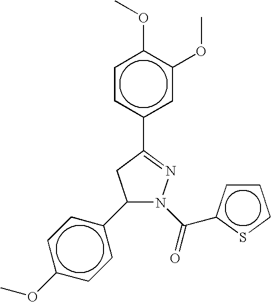

### EXAMPLE 42

### (3-(3,4-dimethoxyphenyl)-5-(4-fluorophenyl)-4,5-dihydro-1H-pyrazol-1-yl)(3,4,5-trimethoxyphenyl)methanone

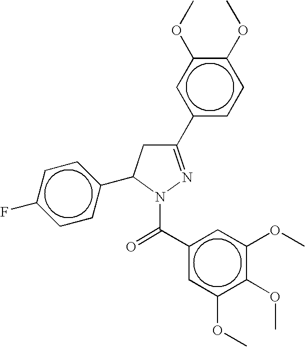

### EXAMPLE 43

### (5-(4-chlorophenyl)-3-(3,4-dimethoxyphenyl)-4,5-dihydro-1H-pyrazol-1-yl)(3,4,5-trimethoxyphenyl)methanone

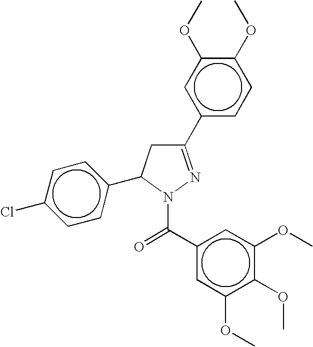

### EXAMPLE 44

### (5-(4-bromophenyl)-3-(3,4-dimethoxyphenyl)-4,5-dihydro-1H-pyrazol-1-yl)(3,4,5-trimethoxyphenyl)methanone

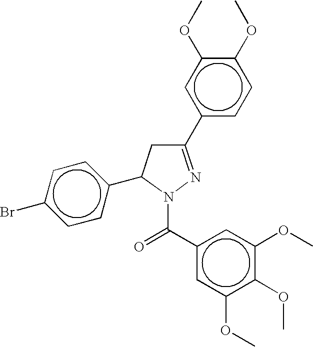

### EXAMPLE 45

### (3-(3,4-dimethoxyphenyl)-5-p-tolyl-4,5-dihydro-1H-pyrazol-1-yl)(3,4,5-trimethoxyphenyl)methanone

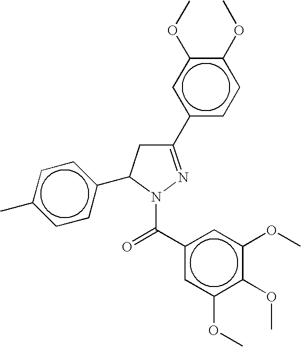

### EXAMPLE 46

### (5-bromofuran-2-yl)(3-(3,4-dimethoxyphenyl)-5-(4-fluorophenyl)-4,5-dihydro-1H-pyrazol-1-yl)methanone

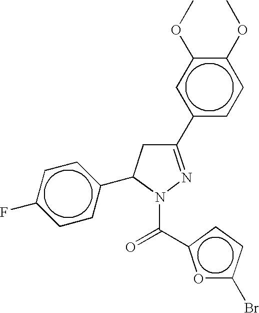

### EXAMPLE 47

### (5-bromofuran-2-yl)(5-(4-chlorophenyl)-3-(3,4-dimethoxyphenyl)-4,5-dihydro-1H-pyrazol-1-yl)methanone

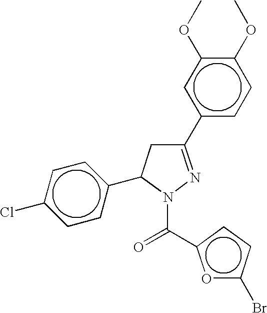

### EXAMPLE 48

### (5-bromofuran-2-yl)(3-(3,4-dimethoxyphenyl)-5-p-tolyl-4,5-dihydro-1H-pyrazol-1-yl)methanone

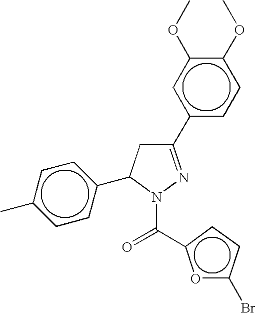

### EXAMPLE 49

### (5-bromofuran-2-yl)(3-(3,4-dimethoxyphenyl)-5-(2-fluorophenyl)-4,5-dihydro-1H-pyrazol-1-yl)methanone

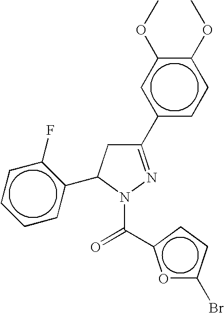

### EXAMPLE 50

### (5-bromofuran-2-yl)(5-(2-chlorophenyl)-3-(3,4-dimethoxyphenyl)-4,5-dihydro-1H-pyrazol-1-yl)methanone

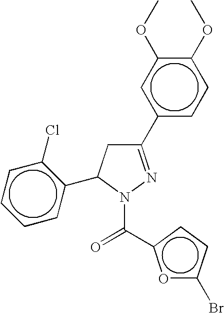

### EXAMPLE 51

### (5-bromofuran-2-yl)(3-(3,4-dimethoxyphenyl)-5-phenyl-4,5-dihydro-1H-pyrazol-1-yl)methanone

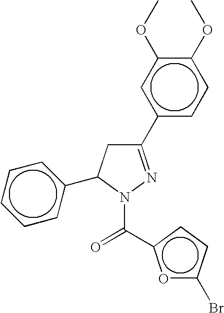

### EXAMPLE 52

### 3-(3,4-dimethoxyphenyl)-5-(3-nitrophenyl)-1-tosyl-4,5-dihydro-1H-pyrazole

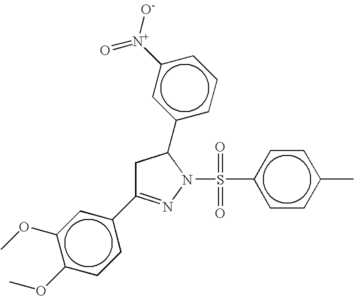

### EXAMPLE 53

### 3-(3,4-dimethoxyphenyl)-5-(3-nitrophenyl)-1-(phenylsulfonyl)-4,5-dihydro-1H-pyrazole

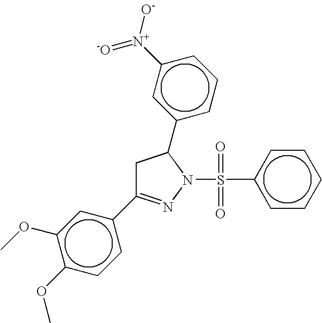

### EXAMPLE 54

### 3-(3,4-dimethoxyphenyl)-5-(4-fluorophenyl)-1-(phenylsulfonyl)-4,5-dihydro-1H-pyrazole

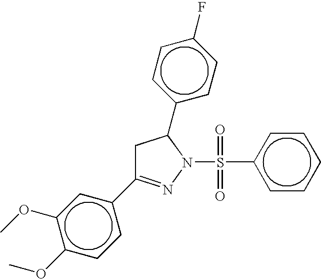

### EXAMPLE 55

### 3-(3,4-dimethoxyphenyl)-5-(4-fluorophenyl)-1-tosyl-4,5-dihydro-1H-pyrazole

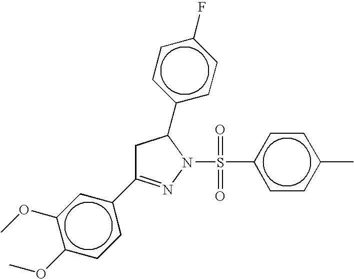

### EXAMPLE 56

### 3-(3,4-dimethoxyphenyl)-1-(phenylsulfonyl)-5-p-tolyl-4,5-dihydro-1H-pyrazole

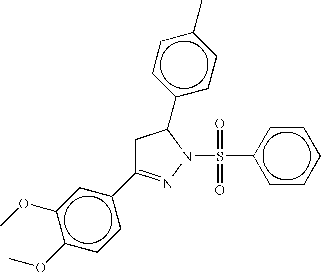

### EXAMPLE 57

### 3-(3,4-dimethoxyphenyl)-5-p-tolyl-1-tosyl-4,5-dihydro-1H-pyrazole

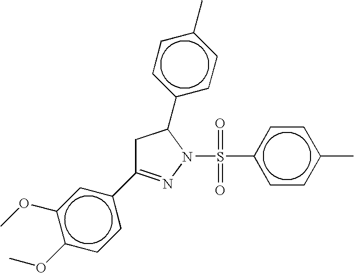

### EXAMPLE 58

### 3-(3,4-dimethoxyphenyl)-5-(2-fluorophenyl)-1-(phenylsulfonyl)-4,5-dihydro-1H-pyrazole

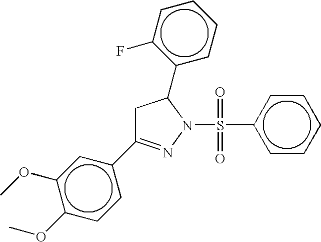

### EXAMPLE 59

### 5-(2-chlorophenyl)-3-(3,4-dimethoxyphenyl)-1-(phenylsulfonyl)-4,5-dihydro-1H-pyrazole

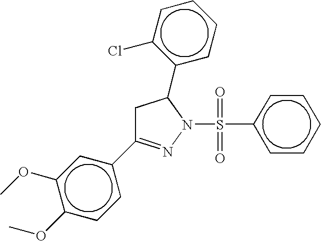

### EXAMPLE 60

### 5-(2-chlorophenyl)-3-(3,4-dimethoxyphenyl)-1-tosyl-4,5-dihydro-1H-pyrazole

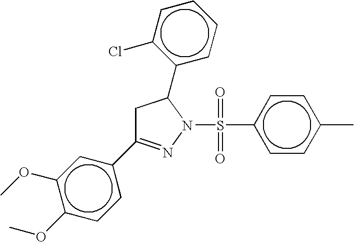

### EXAMPLE 61

### 3-(3,4-dimethoxyphenyl)-5-(2-methoxyphenyl)-1-(phenylsulfonyl)-4,5-dihydro-1H-pyrazole

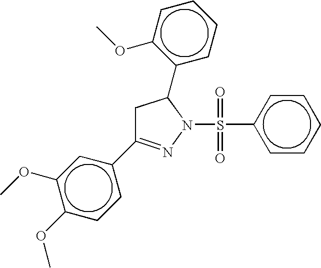

### EXAMPLE 62

### 3-(3,4-dimethoxyphenyl)-5-phenyl-1-tosyl-4,5-dihydro-1H-pyrazole

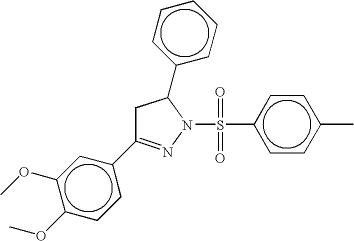

### EXAMPLE 63

### 3-(3,4-dimethoxyphenyl)-5-phenyl-1-(phenylsulfonyl)-4,5-dihydro-1H-pyrazole

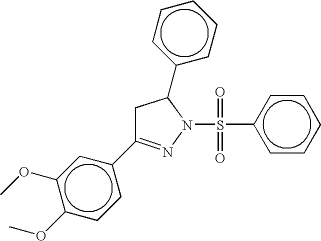

### EXAMPLE 64

### 5-(5-(2,5-dimethoxyphenyl)-3-(3,4-dimethoxyphenyl)-4,5-dihydro-1H-pyrazol-1-yl)-5-oxopentanoic acid

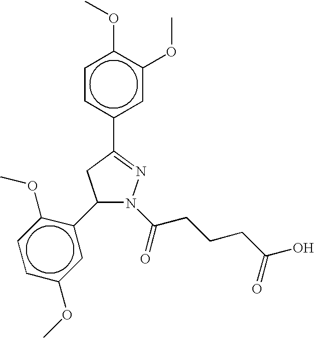

### EXAMPLE 65

### 5-(3-(3,4-dimethoxyphenyl)-5-(4-fluorophenyl)-4,5-dihydro-1H-pyrazol-1-yl)-5-oxopentanoic acid

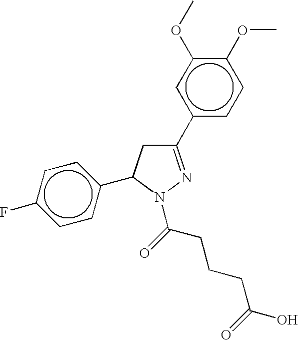

### EXAMPLE 66

### 5-(3-(3,4-dimethoxyphenyl)-5-(4-methoxyphenyl)-4,5-dihydro-1H-pyrazol-1-yl)-5-oxopentanoic acid

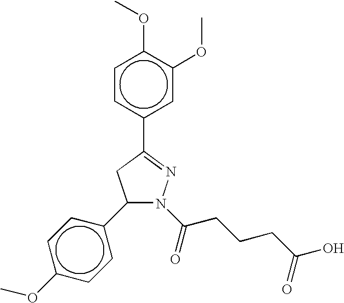

### EXAMPLE 67

### 5-(5-(2-chlorophenyl)-3-(3,4-dimethoxyphenyl)-4,5-dihydro-1H-pyrazol-1-yl)-5-oxopentanoic acid

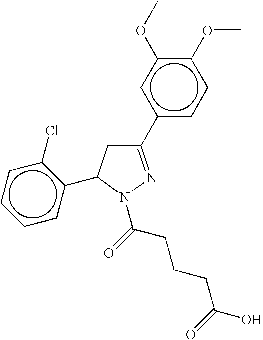

### EXAMPLE 68

### 4-(3,5-bis(3,4-dimethoxyphenyl)-4,5-dihydro-1H-pyrazol-1-yl)-4-oxobutanoic acid

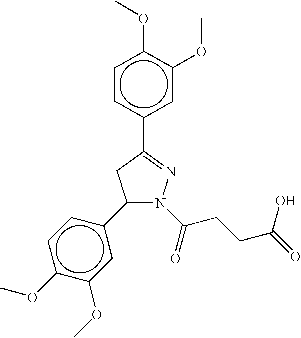

### EXAMPLE 69

### 4-(5-(2,3-dimethoxyphenyl)-3-(3,4-dimethoxyphenyl)-4,5-dihydro-1H-pyrazol-1-yl)-4-oxobutanoic acid

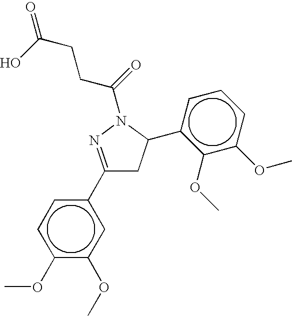

### EXAMPLE 70

### 4-(5-(4-chlorophenyl)-3-(3,4-dimethoxyphenyl)-4,5-dihydro-1H-pyrazol-1-yl)-4-oxobutanoic acid

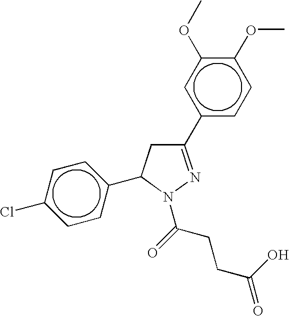

### EXAMPLE 71

### 4-(3-(3,4-dimethoxyphenyl)-5-p-tolyl-4,5-dihydro-1H-pyrazol-1-yl)-4-oxobutanoic acid

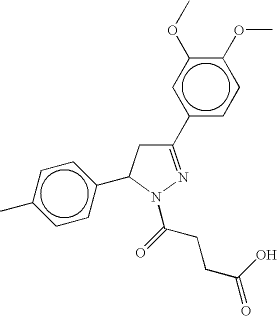

### EXAMPLE 72

### 4-(3-(3,4-dimethoxyphenyl)-5-(4-fluorophenyl)-4,5-dihydro-1H-pyrazol-1-yl)-4-oxobutanoic acid

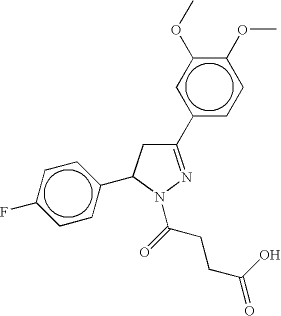

### EXAMPLE 73

### 1-(3-(3,4-dimethoxyphenyl)-5-(1,3-diphenyl-1H-pyrazol-4-yl)-4,5-dihydro-1H-pyrazol-1-yl)ethanone

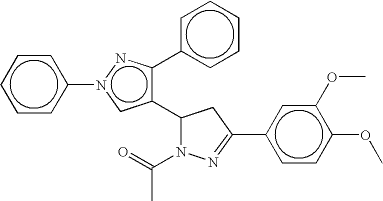

### EXAMPLE 74

### 1-(5-(2-chloro-6-methylquinolin-3-yl)-3-(3,4-dimethoxyphenyl)-4,5-dihydro-1H-pyrazol-1-yl)ethanone

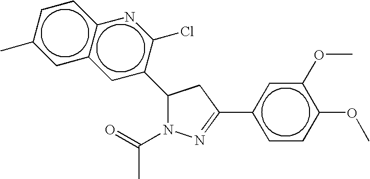

### EXAMPLE 75

### 1-(5-(2-chloro-7-methylquinolin-3-yl)-3-(3,4-dimethoxyphenyl)-4,5-dihydro-1H-pyrazol-1-yl)ethanone

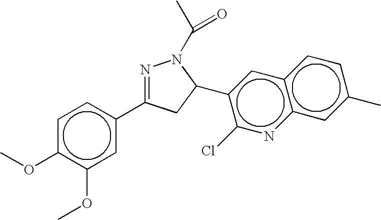

### EXAMPLE 76

### 1-(5-(2-chloro-8-methylquinolin-3-yl)-3-(3,4-dimethoxyphenyl)-4,5-dihydro-1H-pyrazol-1-yl)ethanone

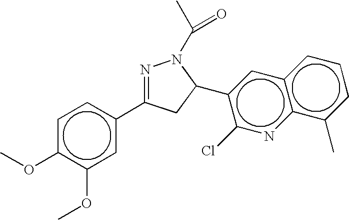

### EXAMPLE 77

### (3-(3,4-dimethoxyphenyl)-5-(3-nitrophenyl)-4,5-dihydro-1H-pyrazol-1-yl)(3,4,5-trimethoxyphenyl)methanone

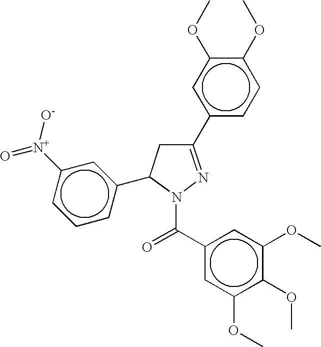

### EXAMPLE 78

### (3,5-bis(3,4-dimethoxyphenyl)-4,5-dihydro-1H-pyrazol-1-yl)(3,4,5-trimethoxyphenyl)methanone

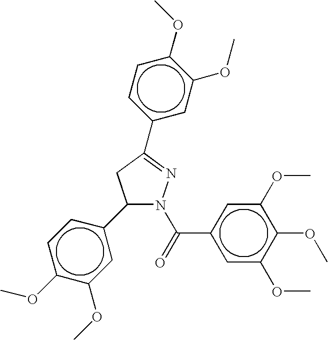

### EXAMPLE 79

### (3-(3,4-dimethoxyphenyl)-5-(2-fluorophenyl)-4,5-dihydro-1H-pyrazol-1-yl)(3,4,5-trimethoxyphenyl)methanone

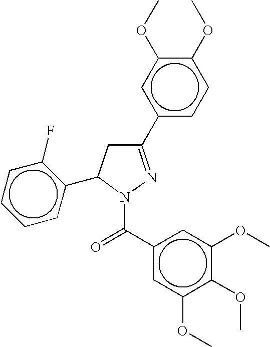

### EXAMPLE 80

### (3-(3,4-dimethoxyphenyl)-5-(2-methoxyphenyl)-4,5-dihydro-1H-pyrazol-1-yl)(3,4,5-trimethoxyphenyl)methanone

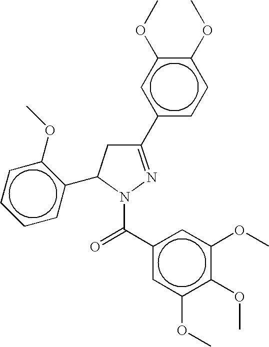

### EXAMPLE 81

### (5-bromofuran-2-yl)(3-(3,4-dimethoxyphenyl)-5-(4-(dimethylamino)phenyl)-4,5-dihydro-1H-pyrazol-1-yl)methanone

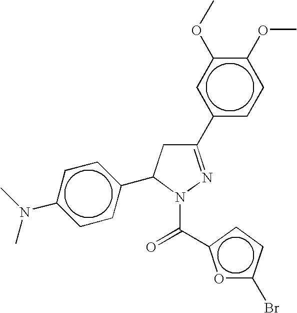

### EXAMPLE 82

### (3,5-bis(3,4-dimethoxyphenyl)-4,5-dihydro-1H-pyrazol-1-yl)(5-bromofuran-2-yl)methanone

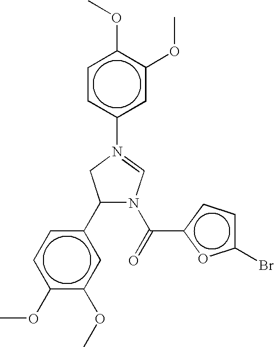

### EXAMPLE 83

### (5-bromofuran-2-yl)(5-(2,5-dimethoxyphenyl)-3-(3,4-dimethoxyphenyl)-4,5-dihydro-1H-pyrazol-1-yl)methanone

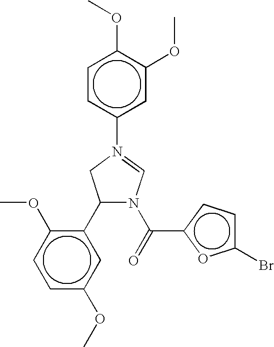

### EXAMPLE 84

### (5-bromofuran-2-yl)(5-(2,3-dimethoxyphenyl)-3-(3,4-dimethoxyphenyl)-4,5-dihydro-1H-pyrazol-1-yl)methanone

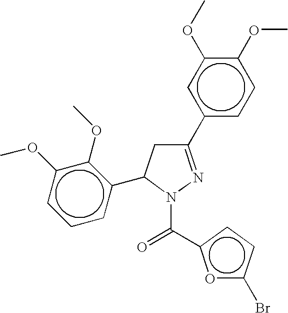

### EXAMPLE 85

### 4-(3-(3,4-dimethoxyphenyl)-1-tosyl-4,5-dihydro-1H-pyrazol-5-yl)-N,N-dimethylaniline

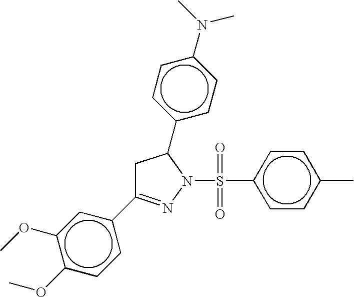

### EXAMPLE 86

### 3,5-bis(3,4-dimethoxyphenyl)-1-(phenylsulfonyl)-4,5-dihydro-1H-pyrazole

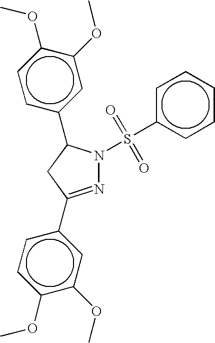

### EXAMPLE 87

### 3,5-bis(3,4-dimethoxyphenyl)-1-tosyl-4,5-dihydro-1H-pyrazole

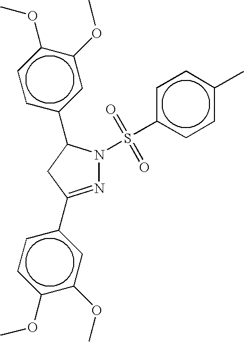

### EXAMPLE 88

### 5-(2,5-dimethoxyphenyl)-3-(3,4-dimethoxyphenyl)-1-(phenylsulfonyl)-4,5-dihydro-1H-pyrazole

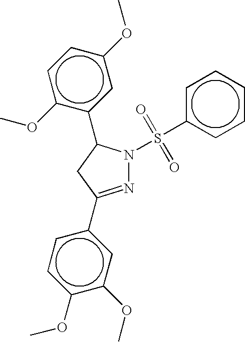

### EXAMPLE 89

### 5-(2,5-dimethoxyphenyl)-3-(3,4-dimethoxyphenyl)-1-tosyl-4,5-dihydro-1H-pyrazole

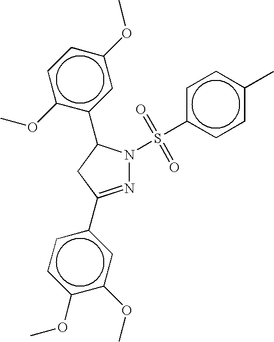

### EXAMPLE 90

### 5-(2,3-dimethoxyphenyl)-3-(3,4-dimethoxyphenyl)-1-(phenylsulfonyl)-4,5-dihydro-1H-pyrazole

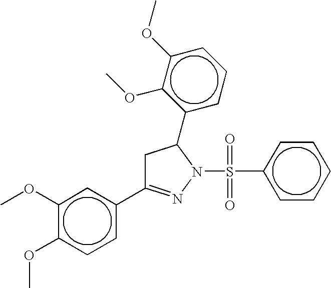

### EXAMPLE 91

### 5-(2,3-dimethoxyphenyl)-3-(3,4-dimethoxyphenyl)-1-tosyl-4,5-dihydro-1H-pyrazole

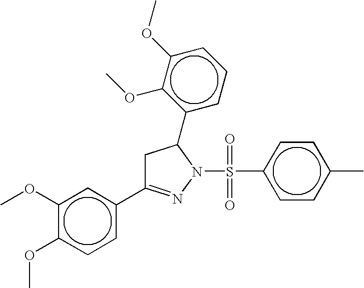

### EXAMPLE 92

### 3-(3,4-dimethoxyphenyl)-5-(4-methoxyphenyl)-1-tosyl-4,5-dihydro-1H-pyrazole

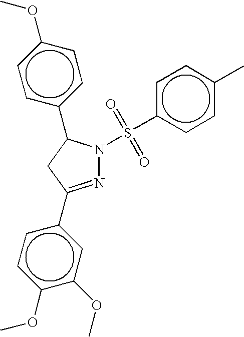

### EXAMPLE 93

### 3-(3,4-dimethoxyphenyl)-5-(2-methoxyphenyl)-1-tosyl-4,5-dihydro-1H-pyrazole

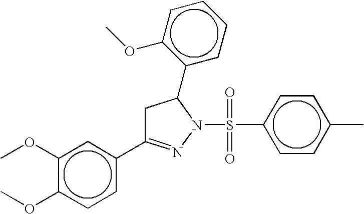

### EXAMPLE 94

### 5-(5-(2,3-dimethoxyphenyl)-3-(3,4-dimethoxyphenyl)-4,5-dihydro-1H-pyrazol-1-yl)-5-oxopentanoic acid

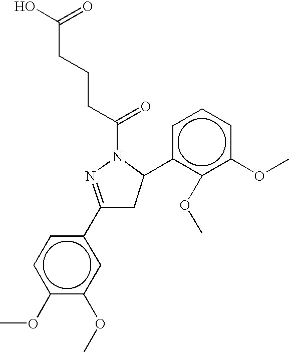

### EXAMPLE 95

### 5-(5-(4-chlorophenyl)-3-(3,4-dimethoxyphenyl)-4,5-dihydro-1H-pyrazol-1-yl)-5-oxopentanoic acid

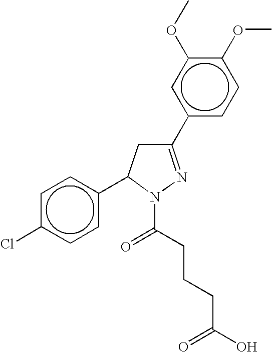

### EXAMPLE 96

### 5-(3-(3,4-dimethoxyphenyl)-5-p-tolyl-4,5-dihydro-1H-pyrazol-1-yl)-5-oxopentanoic acid

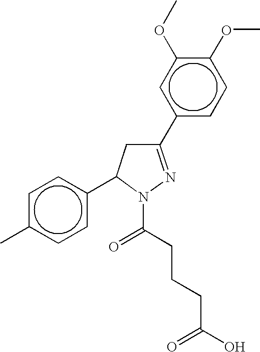

### EXAMPLE 97

### 4-(3-(3,4-dimethoxyphenyl)-5-(4-methoxyphenyl)-4,5-dihydro-1H-pyrazol-1-yl)-4-oxobutanoic acid

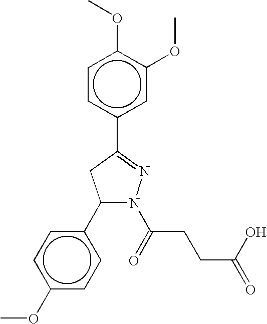

### EXAMPLE 98

### 4-(3-(3,4-dimethoxyphenyl)-5-(2-methoxyphenyl)-4,5-dihydro-1H-pyrazol-1-yl)-4-oxobutanoic acid

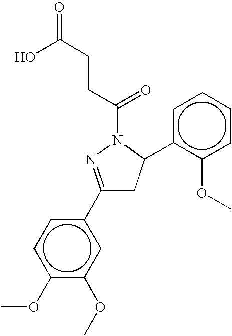

### EXAMPLE 99

### 4-(3-(3,4-dimethoxyphenyl)-5-phenyl-4,5-dihydro-1H-pyrazol-1-yl)-4-oxobutanoic acid

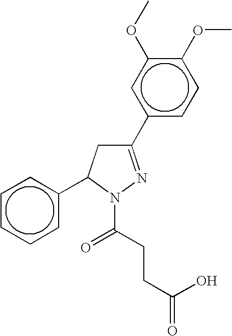

### EXAMPLE 100

### 1-(3,5-bis(3,4-dimethoxyphenyl)-4,5-dihydro-1H-pyrazol-1-yl)ethanone

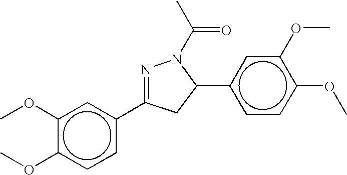

### EXAMPLE 101

### 1-(5-(2,3-dimethoxyphenyl)-3-(3,4-dimethoxyphenyl)-4,5-dihydro-1H-pyrazol-1-yl)ethanone

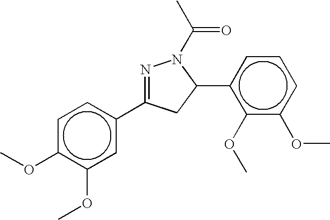

### EXAMPLE 102

### 1-(3-(3,4-dimethoxyphenyl)-5-(4-fluorophenyl)-4,5-dihydro-1H-pyrazol-1-yl)ethanone

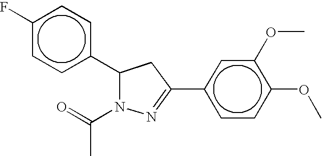

### EXAMPLE 103

### 1-(3-(3,4-dimethoxyphenyl)-5-(4-methoxyphenyl)-4,5-dihydro-1H-pyrazol-1-yl)ethanone

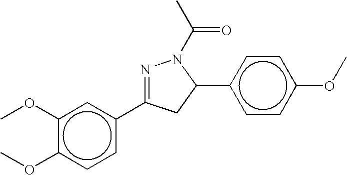

### EXAMPLE 104

### 1-(3-(3,4-dimethoxyphenyl)-5-(2-fluorophenyl)-4,5-dihydro-1H-pyrazol-1-yl)ethanone

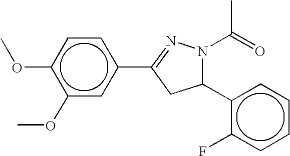

### EXAMPLE 105

### 1-(3-(3,4-dimethoxyphenyl)-5-(2-methoxyphenyl)-4,5-dihydro-1H-pyrazol-1-yl)ethanone

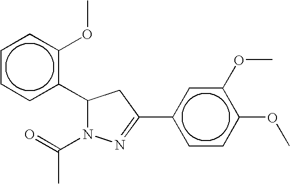

### EXAMPLE 106

### 1-(3-(3,4-dimethoxyphenyl)-5-phenyl-4,5-dihydro-1H-pyrazol-1-yl)ethanone

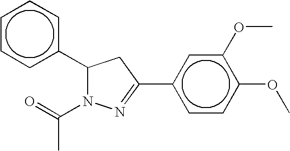

### Biological Activity Assay

The activity of the compounds in Examples 1-106 as PDE4 inhibitors is illustrated in the following assays.

### PDE4 IMAP Assay

Compounds may be screened for functional potency in in vitro biochemical assays for their ability to inhibit the activity of phosphodiesterase (PDE) enzymes. A commercial PDE assay, Molecular Device's PDE IMAP assay, was modified and utilized to measure the inhibitory activity of compounds on PDE isoforms. The IMAP technology is based on the high affinity binding of phosphate by immobilized metal (MIII) coordination complexes on nanoparticles. This IMAP “binding reagent” complexes with phosphate groups on the nucleotide monophosphates generated in the reaction. Such binding causes a change in the rate of the molecular motion of the nucleotide, and results in an increase in the fluorescence polarization value observed for the fluorescent label attached to the substrate. PDE inhibitors prevent the generation of nucleotide monophosphates and the subsequent increase in polarization value observed for the fluorescent label attached to the substrate. The catalytic domain of PDE4B was fused to an amino-terminus hex-histadine affinity tag and purified. PDE4B enzyme was incubated with compounds for 10 minutes. Fluorescently labeled cyclic AMP substrates were then added to the PDE enzyme mix and incubated for 2 hours at room temperature. IMAP binding reagent was then added to the enzyme mix, incubated for 10-30 minutes and fluorescent polarization of the fluorescently labeled substrate was read either with the Perkin Elmer Viewlux or Molecular Devices Acquest or Analyst. Roflumilast and Rolipram are the positive controls used in the assay.

### PDE4 Cellular CNG Assay

Compounds may be screened for functional potency in in vitro cellular assays for their ability to inhibit the activity of phosphodiesterase (PDE) enzymes. A commercial membrane potential assay, Molecular Device's membrane potential assay kit, was modified and utilized to measure the inhibitory activity of compounds on PDE isoforms in living cells. The cellular assay utilizes genetically engineered cyclic nucleotide-gated (CNG) channels as cyclic-AMP (cAMP) sensors. On binding cAMP these ion channels open, allowing the flow of cations across the plasma membrane. CNG channel activity can be monitored by measuring Ca²⁺influx in cell populations. The influx of Ca causes a change in membrane potential that is measured by voltage sensitive dyes. PDE inhibitors prevent the degradation of cAMP that is generated by adenylyl cyclases that are activated upon stimulation of G protein-coupled receptors (GPCR) linked to the cAMP pathway. In brief, Molecular Device's membrane potential dye was added to HEK293 cells stably expressing the CNG channel and incubated for 2 hours at room temperature. Test compounds were then added to the cells and incubated for 15 minutes. A GPCR agonist was then added to the cells to stimulate the cAMP pathway and incubated for 45 minutes. Fluorescent measurements were then conducted with either the Molecular Devices Acquest or Analyst. PDE inhibitors will inhibit the degradation of cAMP and cause an increase in fluorescent signal. Roflumilast and Rolipram are the positive controls used in the assay.

The following are examples of compounds and assay data disclosed by the present invention.

TABLE 1
Biological Activity

| | | PDE4 Cellular CNG Assay |
|---|---|---|
| | PDE4 IMAP Assay | + indicates Activity ≧50% at |
| | + indicates Activity ≧50% at | 10 μM |
| | 10 μM | − indicates Activity <50% at |
| | − indicates Activity <50% at | 10 μM |
| Example | 10 μM | NT indicates Not Tested |
| 1 | − | NT |
| 2 | − | NT |
| 3 | − | NT |
| 4 | − | NT |
| 5 | − | NT |
| 6 | − | NT |
| 7 | − | NT |
| 8 | − | NT |
| 9 | − | NT |
| 10 | − | NT |
| 11 | − | NT |
| 12 | − | NT |
| 13 | − | NT |
| 14 | − | NT |
| 15 | − | NT |
| 16 | − | NT |
| 17 | − | NT |
| 18 | − | NT |
| 19 | − | NT |
| 20 | − | NT |
| 21 | − | NT |
| 22 | − | NT |
| 23 | − | NT |
| 24 | − | NT |
| 25 | − | NT |
| 26 | − | NT |
| 27 | − | NT |
| 28 | − | NT |
| 29 | − | NT |
| 30 | − | NT |
| 31 | + | + |
| 32 | − | NT |
| 33 | + | NT |
| 34 | + | NT |
| 35 | − | NT |
| 36 | + | NT |
| 37 | − | NT |
| 38 | + | NT |
| 39 | − | NT |
| 40 | + | − |
| 41 | + | NT |
| 42 | + | + |
| 43 | + | NT |
| 44 | + | NT |
| 45 | + | NT |
| 46 | + | NT |
| 47 | + | NT |
| 48 | + | NT |
| 49 | + | NT |
| 50 | − | NT |
| 51 | + | NT |
| 52 | − | NT |
| 53 | − | NT |
| 54 | − | NT |
| 55 | − | NT |
| 56 | − | NT |
| 57 | − | NT |
| 58 | − | NT |
| 59 | − | NT |
| 60 | − | NT |
| 61 | − | NT |
| 62 | − | NT |
| 63 | − | NT |
| 64 | − | NT |
| 65 | − | NT |
| 66 | − | NT |
| 67 | − | NT |
| 68 | − | NT |
| 69 | − | NT |
| 70 | − | NT |
| 71 | − | NT |
| 72 | − | NT |
| 73 | − | NT |
| 74 | − | NT |
| 75 | − | NT |
| 76 | − | NT |
| 77 | + | + |
| 78 | − | NT |
| 79 | + | NT |
| 80 | − | NT |
| 81 | − | NT |
| 82 | − | NT |
| 83 | − | NT |
| 84 | − | NT |
| 85 | − | NT |
| 86 | + | NT |
| 87 | − | NT |
| 88 | − | NT |
| 89 | − | NT |
| 90 | − | NT |
| 91 | − | NT |
| 92 | − | NT |
| 93 | − | NT |
| 94 | − | NT |
| 95 | − | NT |
| 96 | − | NT |
| 97 | − | NT |
| 98 | − | NT |
| 99 | − | NT |
| 100 | − | NT |
| 101 | − | NT |
| 102 | + | NT |
| 103 | − | NT |
| 104 | − | NT |
| 105 | − | NT |
| 106 | − | NT |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of inhibition of PDE4 comprising contacting PDE4 with a compound of structural Formula I 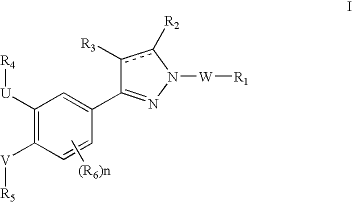
or a salt, ester, or prodrug thereof, wherein:
W is selected from the group consisting of lower alkylene, C(═O), S(═O), SO₂, and null;
U and V are each independently selected from the group consisting of O, S, NR⁷, S(═O), and SO₂;
R¹ is selected from the group consisting of hydrogen, alkenyl, alkoxy, alkyl, alkynyl, amino, aryl, aryloxy, carboxyalkyl, cycloalkoxy, cycloalkyl, heteroaryl, heterocycloalkoxy, heterocycloalkyl, and mercaptyl, any of which may be optionally substituted;
R² is selected from the group consisting of acyl, alkenyl, alkyl, alkynyl, amido, aryl, carboxyl, cyano, cycloalkyl, ester, heteroaryl, heterocycloalkyl, sulfonate, sulfinyl, sulfonyl, and S-sulfonamido, any of which may be optionally substituted;
R³ is selected from the group consisting of hydrogen, acyl, acylamino, acyloxy, alkoxy, alkyl, amino, N-carbamyl, O-carbamyl, hydroxy, and mercaptyl, any of which may be optionally substituted;
R⁴ is selected from the group consisting of acyl, alkenyl, alkyl, alkynyl, aryl, cycloalkyl, cycloalkylalkyl, heteroaryl, and heterocycloalkyl, any of which may be optionally substituted;
R⁵ is selected from the group consisting of alkyl, haloalkyl, and perhaloalkyl, any of which may be optionally substituted;
each R⁶ is independently selected from the group consisting of alkoxy, alkyl, cycloalkoxy, halogen, hydroxy, mercaptyl, nitro, sulfonate, sulfinyl, and sulfonyl, any of which may be optionally substituted;
R⁷ is selected from the group consisting of hydrogen and optionally substituted lower alkyl; and
n is an integer from 0 to 3.

2. The method as recited in claim 1, wherein said compound is of structural Formula II 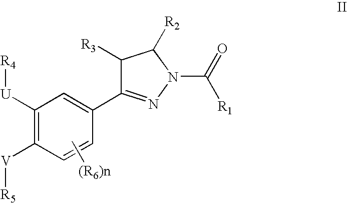
or a salt, ester, or prodrug thereof, wherein:
U and V are each independently selected from the group consisting of O and S;
R¹ is selected from the group consisting of hydrogen, alkenyl, alkyl, alkynyl, aryl, carboxyalkyl, cycloalkyl, heteroaryl, and heterocycloalkyl, any of which may be optionally substituted;
R² is selected from the group consisting of aryl and heteroaryl, either of which may be optionally substituted;
R³ is selected from the group consisting of hydrogen and optionally substituted lower alkyl;
R⁴ and R⁵ are independently selected from the group consisting of alkyl, haloalkyl, and perhaloalkyl, or R⁴ and R⁵ may combine to form a heterocyclic ring system selected from the group consisting of 1,3-dioxolane, 1,4-dioxane, 1,3-oxathiolane, 1,4-oxathiane, 1,3-dithiolane, and 1,4-dithiane, any of which may be optionally substituted by lower alkyl or halogen;
each R⁶ is independently selected from the group consisting of alkoxy, alkyl, halogen, hydroxy, and mercaptyl, any of which may be optionally substituted; and
n is an integer from 0 to 3.

3. The method as recited in claim 2, wherein R² is optionally substituted phenyl.

4. The method as recited in claim 2, wherein said compound is of structural Formula III 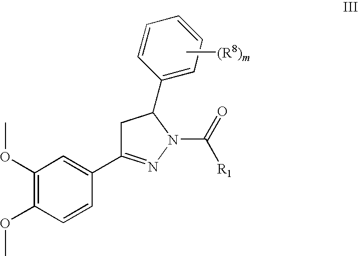
or a salt, ester, or prodrug thereof, wherein:
R¹ is selected from the group consisting of alkyl, carboxyalkyl, heteroaryl, and aryl, any of which may be optionally substituted;
each R⁸ is independently selected from the group consisting of alkoxy, halogen, amino, nitro and alkyl, any of which may be optionally substituted; and
m is an integer from 0 to 2.

5. The method as recited in claim 4 wherein said PDE4 is the PDE4B subtype.

6. The method as recited in claim 1, wherein said compound is selected from the group consisting of Examples 1 to 106.

7. A method of treatment of a PDE4-mediated disease, in a patient in need of such treatment, comprising the administration of a therapeutically effective amount of a compound of Formula I 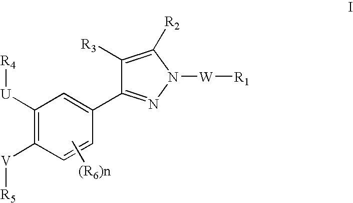
or a salt, ester, or prodrug thereof, wherein:
W is selected from the group consisting of lower alkylene, C(═O), S(═O), SO₂, and null;
U and V are each independently selected from the group consisting of O, S, NR⁷, S(═O), and SO₂;
R¹ is selected from the group consisting of hydrogen, alkenyl, alkoxy, alkyl, alkynyl, amino, aryl, aryloxy, carboxyalkyl, cycloalkoxy, cycloalkyl, heteroaryl, heterocycloalkoxy, heterocycloalkyl, and mercaptyl, any of which may be optionally substituted;
R² is selected from the group consisting of acyl, alkenyl, alkyl, alkynyl, amido, aryl, carboxyl, cyano, cycloalkyl, ester, heteroaryl, heterocycloalkyl, sulfonate, sulfinyl, sulfonyl, and S-sulfonamido, any of which may be optionally substituted;
R³ is selected from the group consisting of hydrogen, acyl, acylamino, acyloxy, alkoxy, alkyl, amino, N-carbamyl, O-carbamyl, hydroxy, and mercaptyl, any of which may be optionally substituted;
R⁴ is selected from the group consisting of acyl, alkenyl, alkyl, alkynyl, aryl, cycloalkyl, cycloalkylalkyl, heteroaryl, and heterocycloalkyl, any of which may be optionally substituted;
R⁵ is selected from the group consisting of alkyl, haloalkyl, and perhaloalkyl, any of which may be optionally substituted;
each R⁶ is independently selected from the group consisting of alkoxy, alkyl, cycloalkoxy, halogen, hydroxy, mercaptyl, nitro, sulfonate, sulfinyl, and sulfonyl, any of which may be optionally substituted;
R⁷ is selected from the group consisting of hydrogen and optionally substituted lower alkyl; and
n is an integer from 0 to 3.

8. The method as recited in claim 7, wherein said compound is of structural Formula II 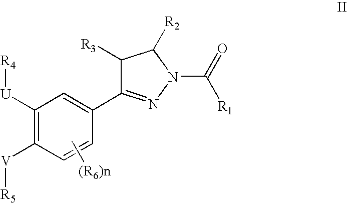
or a salt, ester, or prodrug thereof, wherein:
U and V are each independently selected from the group consisting of O and S;
R¹ is selected from the group consisting of hydrogen, alkenyl, alkyl, alkynyl, aryl, carboxyalkyl, cycloalkyl, heteroaryl, and heterocycloalkyl, any of which may be optionally substituted;
R² is selected from the group consisting of aryl and heteroaryl, either of which may be optionally substituted;
R³ is selected from the group consisting of hydrogen and optionally substituted lower alkyl;
R⁴ and R⁵ are independently selected from the group consisting of alkyl, haloalkyl, and perhaloalkyl, or R⁴ and R⁵ may combine to form a heterocyclic ring system selected from the group consisting of 1,3-dioxolane, 1,4-dioxane, 1,3-oxathiolane, 1,4-oxathiane, 1,3-dithiolane, and 1,4-dithiane, any of which may be optionally substituted by lower alkyl or halogen;
R⁶ is selected from the group consisting of alkoxy, alkyl, halogen, hydroxy, and mercaptyl, any of which may be optionally substituted; and
n is an integer from 0 to 3.

9. The method as recited in claim 8, wherein said PDE4-mediated disease is an ophthalmic disease.

10. The method as recited in claim 9 wherein said ophthalmic disease is selected from the group consisting of dry eye, glaucoma, corneal neovascularization, optic neuritis, Sjogren's syndrome, retinal ganglion degeneration, ocular ischemia, retinitis, retinopathy, uveitis, ocular photophobia, and inflammation and pain associated with acute injury to the eye tissue.

11. The method as recited in claim 8, wherein R² is optionally substituted phenyl.

12. The method as recited in claim 8, wherein said compound is of structural Formula III 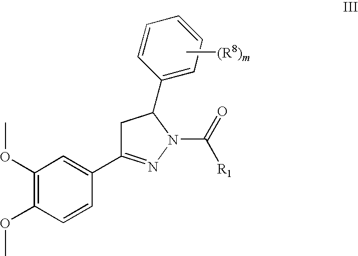
or a salt, ester, or prodrug thereof, wherein:
R¹ is selected from the group consisting of alkyl, carboxyalkyl, heteroaryl, and aryl, any of which may be optionally substituted;
R⁸ is selected from the group consisting of alkoxy, halogen, amino, nitro and alkyl, any of which may be optionally substituted; and
m is an integer from 0 to 2.

13. The method as recited in claim 8 wherein said PDE4 is the PDE4B subtype.

14. The method as recited in claim 7, wherein said compound is selected from the group consisting of Examples 1 to 106.

15. A compound for use as a medicament, having structural Formula II 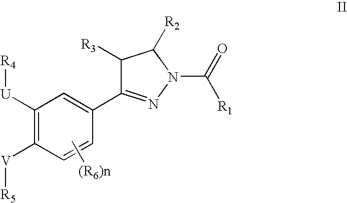
or a salt, ester, or prodrug thereof, wherein:
U and V are each independently selected from the group consisting of O and S;
R¹ is selected from the group consisting of hydrogen, alkenyl, alkyl, alkynyl, aryl, carboxyalkyl, cycloalkyl, heteroaryl, and heterocycloalkyl, any of which may be optionally substituted;
R² is selected from the group consisting of aryl and heteroaryl, either of which may be optionally substituted;
R³ is selected from the group consisting of hydrogen and optionally substituted lower alkyl;
R⁴ and R⁵ are independently selected from the group consisting of alkyl, haloalkyl, and perhaloalkyl, or R⁴ and R⁵ may combine to form a heterocyclic ring system selected from the group consisting of 1,3-dioxolane, 1,4-dioxane, 1,3-oxathiolane, 1,4-oxathiane, 1,3-dithiolane, and 1,4-dithiane, any of which may be optionally substituted by lower alkyl or halogen;
each R⁶ is independently selected from the group consisting of alkoxy, alkyl, halogen, hydroxy, and mercaptyl, any of which may be optionally substituted; and
n is an integer from 0 to 3.

16. A compound for use in the manufacture of a medicament for the prevention or treatment of a disease or condition ameliorated by the inhibition of PDE4, having structural Formula II 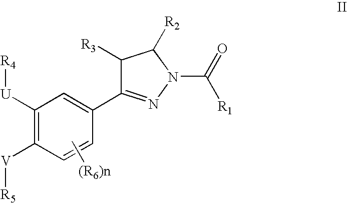
or a salt, ester, or prodrug thereof, wherein:
U and V are each independently selected from the group consisting of O and S;
R¹ is selected from the group consisting of hydrogen, alkenyl, alkyl, alkynyl, aryl, carboxyalkyl, cycloalkyl, heteroaryl, and heterocycloalkyl, any of which may be optionally substituted;
R² is selected from the group consisting of aryl and heteroaryl, either of which may be optionally substituted;
R³ is selected from the group consisting of hydrogen and optionally substituted lower alkyl;
R⁴ and R⁵ are independently selected from the group consisting of alkyl, haloalkyl, and perhaloalkyl, or R⁴ and R⁵ may combine to form a heterocyclic ring system selected from the group consisting of 1,3-dioxolane, 1,4-dioxane, 1,3-oxathiolane, 1,4-oxathiane, 1,3-dithiolane, and 1,4-dithiane, any of which may be optionally substituted by lower alkyl or halogen;
each R⁶ is independently selected from the group consisting of alkoxy, alkyl, halogen, hydroxy, and mercaptyl, any of which may be optionally substituted; and
n is an integer from 0 to 3.

17. A pharmaceutical composition comprising a pharmaceutically acceptable carrier together with a compound of structural Formula II 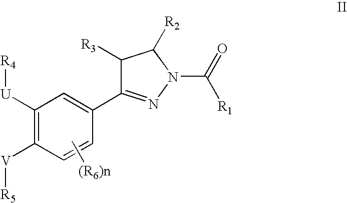
or a salt, ester, or prodrug thereof, wherein:
U and V are each independently selected from the group consisting of O and S;
R¹ is selected from the group consisting of hydrogen, alkenyl, alkyl, alkynyl, aryl, carboxyalkyl, cycloalkyl, heteroaryl, and heterocycloalkyl, any of which may be optionally substituted;
R² is selected from the group consisting of aryl and heteroaryl, either of which may be optionally substituted;
R³ is selected from the group consisting of hydrogen and optionally substituted lower alkyl;
R⁴ and R⁵ are independently selected from the group consisting of alkyl, haloalkyl, and perhaloalkyl, or R⁴ and R⁵ may combine to form a heterocyclic ring system selected from the group consisting of 1,3-dioxolane, 1,4-dioxane, 1,3-oxathiolane, 1,4-oxathiane, 1,3-dithiolane, and 1,4-dithiane, any of which may be optionally substituted by lower alkyl or halogen;
each R⁶ is independently selected from the group consisting of alkoxy, alkyl, halogen, hydroxy, and mercaptyl, any of which may be optionally substituted; and
n is an integer from 0 to 3.

18. The pharmaceutical composition as recited in claim 17, useful for the treatment or prevention of a PDE4-mediated disease.

19. The pharmaceutical composition as recited in claim 18 wherein said PDE4 is the PDE4B subtype.

20. A method of treatment of a PDE4-mediated disease comprising the administration of:
a. a therapeutically effective amount of a compound of structural Formula II 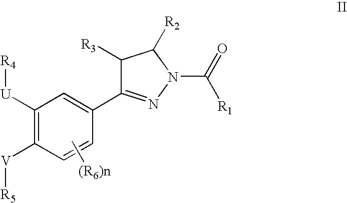
or a salt, ester, or prodrug thereof, wherein:
U and V are each independently selected from the group consisting of O and S;
R¹ is selected from the group consisting of hydrogen, alkenyl, alkyl, alkynyl, aryl, carboxyalkyl, cycloalkyl, heteroaryl, and heterocycloalkyl, any of which may be optionally substituted;
R² is selected from the group consisting of aryl and heteroaryl, either of which may be optionally substituted;
R³ is selected from the group consisting of hydrogen and optionally substituted lower alkyl;
R⁴ and R⁵ are independently selected from the group consisting of alkyl, haloalkyl, and perhaloalkyl, or R⁴ and R⁵ may combine to form a heterocyclic ring system selected from the group consisting of 1,3-dioxolane, 1,4-dioxane, 1,3-oxathiolane, 1,4-oxathiane, 1,3-dithiolane, and 1,4-dithiane, any of which may be optionally substituted by lower alkyl or halogen;
each R⁶ is independently selected from the group consisting of alkoxy, alkyl, halogen, hydroxy, and mercaptyl, any of which may be optionally substituted; and
n is an integer from 0 to 3; and
b. another therapeutic agent.

21. The method as recited in claim 20 wherein said PDE4 is the PDE4B subtype.
